(19) United States
(12) Patent Application Publication
(10) Pub. No.: US 2006/0217670 A1
(43) Pub. Date: Sep. 28, 2006

(54) Assembly for aspiration and/or dispensing of organisms and fluids

(75) Inventors: Michael D. Cecchi, Madison, CT (US); Monica Mezezi, Guelph (CA)

Correspondence Address: William W. Jones, 6 Juniper Lane, Madison, CT, 06443, US

(73) Assignee: Genx International, Inc.

(21) Appl. No.: 11/089,633

(22) Filed: Mar. 28, 2005

(51) Int. Cl. A61M 5/00

(52) U.S. Cl. 604/209; 604/218; 604/227

(57) ABSTRACT

An assembly for the drawing or dispensing of materials by utilizing a gear or gear system provides an ergonomically superior device. The gear system in the assembly may allow the assembly to dispense materials or create a vacuum for multiple tasks at the same time. The materials may be dispensed in various or different volumes simultaneously by the assembly. The assembly will also allow the creation of a vacuum and dispensing of fluids at the same time. The assembly will enable dispensing fluids in various ratios or creating a vacuum or pressure, or both simultaneously. The assembly results in a more ergonomically suitable syringe and/or dispenser for the user.

### TECHNICAL FIELD

This invention relates to an assembly, for the dispensing of fluids or creation of a vacuum of differing and various ratios. More particularly, the assembly of this invention is especially useful for dispensing of small amounts of fluids, or for creating a vacuum, from or in a small volume catheter or pipette.

### BACKGROUND ART

Syringes are generally used either to disperse liquids or to create a vacuum. In the area of in vitro fertilization in human and animal reproduction, syringes are use to create a small volume at the end of IUI or embryo transfer catheters, and to pull the culture media products or specimens into the syringe or into a length of tubing, such as a pipette or catheter, which may be attached to the end of the syringe.

The problem presented by the attachment of a syringe to the end of embryo transfer catheters is that the inner walls of the catheters are only 0.60 mm in diameter. The individual using these catheters in combination with currently available syringes, generally use a one cc syringe, which is typically the smallest disposable syringe available. The problem that is encountered results from the fact that the volume of the syringe is approximately fifty times the volume of the embryo transfer catheters. The protocol for embryo transfer suggests that the embryos be drawn approximately one centimeter into the distal part of the catheter. The syringe must therefore be drawn back to a degree which will be operative to cause the catheter to be filled one cm. Great skill is presently required in accomplishing such precision of movement. Due to the significant difference in the volume of the syringe and the volume of the catheter, the technician can only draw the syringe back one eighth of an inch. This is very difficult to do accurately. It also means the technician cannot accurately or ergonomically control the amount of vacuum that he or she must create.

Another problem relates to the fact that it is very difficult to make a syringe that would have a volume and length of pull that is equal to the volume and desired draw depth of the catheters. If the inner bore size of the syringe cavity would be the same as the catheters (0.60 mm) and would thus require a plunger of a very small diameter, this small diameter plunger would be very difficult to manufacture so as to hold the tight tolerances necessary. Thus it would be very difficult and expensive to produce a workable syringe of the necessary smaller size which would be able to hold the necessary vacuum.

In the prior art, syringes have been used to deliver multiple ingredients at the same time. U.S. Pat. No. 5,290,259 Fisher shows a delivery system for holding a pair of syringes, to allow the simultaneous delivery of two or more substances.

U.S. Pat. No. 5,314,412 Rex discloses the use of an apparatus to simultaneously inject two different kinds of preparations in preset proportions utilizing two syringe barrels.

U.S. Pat. No. 5,116,315 Capozzi discloses a biological syringe system to deliver a first and second fluid in a mixed solution size.

U.S. Pat. No. 5,445,614 Haber discloses a unitary cavity syringe with two plungers to deliver two different components.

It would be desirable to create a device which would allow the user to draw a very small sample or to create a very small volume more accurately and ergonomically.

### DISCLOSURE OF THE INVENTION

This invention relates to an improved syringe assembly that utilizes gears or sets of gears. The syringe of this invention includes gears, a plunger, a thumb actuator assembly and an outer syringe-shaped housing. By using gears or sets of gears as a link between the thumb actuator and the plunger, the syringe can be used to draw or dispense fluids, and to draw a vacuum or create pressure when the thumb assembly and plunger assembly are drawn at different ratios, rates, or lengths. In one embodiment of the syringe assembly, a gear or gear set serves as the link and allows the drawing of a small vacuum at the plunger end of the assembly by moving the plunger a short distance while the thumb assembly is moved a longer more easily ergonomically attainable distance, which enables greater accuracy and ease of use of the syringe assembly.

Instead of using a toothed gear link in the housing, one could use a compound diameter cylindrical rotatable link, one cylindrical portion of which has a greater diameter than another cylindrical portion or portions thereof. The larger diameter cylindrical portion will thus have a greater circumference than the smaller diameter cylindrical portion or portions. The circumference of each of the portions can be provided with gear teeth or high friction surfaces which will serve as driving connections between axially movable components of the syringe, which components are described in greater detail below.

As noted above the syringe assembly has an outer housing component in which the internal components of the assembly are disposed. The front portion of the housing has a frontal bore which is sized to produce a predetermined vacuum and is of a size that will be in a desired proportion to the diameter of an aspiration catheter, or other adjunct device. A plunger is disposed in the frontal bore of the housing and is sized to create an airtight seal with the inner wall of the frontal bore. When the link is a toothed gear, the rear portion of the plunger has a toothed rack extension molded or cut into it. The rack teeth mesh with the gear-toothed link, which is disposed in the housing. The rear thumb part also has a toothed rack extension molded or cut therein to mesh with the aforesaid gear-toothed link. The area where the thumb is used can be flat or can have a thumb ring. When a non-gear-toothed link is used, the extensions of the thumb piece and the plunger will not be provided with gear teeth, but will be provided instead with high coefficient of friction surfaces which engage the high coefficient of friction surfaces on the rotatable link.

In one embodiment of the device, the front portion cavity can have a 0.090″ inner diameter, which is greater than the 0.060″ inner diameter of an aspiration catheter to which the syringe assembly is connected. The extension portion of the plunger engages the link assembly to allow the plunger assembly to move in synchronization with the link. The subassembly parts will be inserted into a body assembly to complete the unit. The thumb portion extension also engages the link, so that linear movement of the thumb portion of the assembly rotates the link which in turn causes linear movement of the plunger component of the assembly. The link will have different diameter components, so as to result in the thumb and plunger parts of the assembly moving different distances when the thumb part is either pulled or pushed. For example, the operator may draw on the thumb assembly for a distance of three centimeters. The thumb assembly would then turn the first portion of the link it is in contact with. This first portion of the link is attached to or is in contact with a second portion of the link which has a smaller diameter than the first portion. As the first portion turns, it, in turn, turns the second portion. The second portion in turn moves the plunger in the front of the device so as to create suction in the front cavity. This in turn will create suction in the attached tubing or catheter, which will in turn draw air or fluids into the catheter, accomplishing what it was set out to do. Thus, depending on the ratio of the link portion diameters, the extent of movement of the plunger, and thus the positive or negative pressure created thereby, will be a fraction of the extent of movement of the thumb part of the assembly. Thus an easily accomplished extent of movement of the thumb piece will result in a smaller extent of movement of the plunger which would be difficult to accomplish with a one to one movement ratio between the thumb part and the plunger part. Thus, a small degree of suction can be applied to the catheter by the plunger as the result of an ergonomically easily accomplished thumb part stroke.

The assembly of this invention will allow the making of syringe assemblies that will be usable for applications such as the transferring of embryos or sperm. The advantage is that the technician may draw the thumb assembly a workable ergonomic distance, such as three centimeters, to, in turn draw the frontal plunger assembly only one centimeter. The size of the plunger diameter can also differ from that of the thumb assembly and that of the catheter.

The assembly of this invention may be used for the aspiration of embryos, cells, or tissues during procedures for transferring the aspirated material from on site to another. The catheter, pipette or microtool may be attached to the assembly of this invention in at least two different ways. One way involves the catheter or pipette being attached to the front tip portion of the assembly of this invention by means of a friction fit with the outer portion of the tip of the assembly, which may be involve the use of a standard luer lock fitting. A second way to attach a pipette would be to insert the pipette into the bore of the tip of the assembly. The second way would be preferred for the attachment of microtools or pipettes where the outer diameter of these attachments may be as small as 300 μm in diameter. Thus, the assembly of this invention may be used in micro aspiration of fluids or organisms into micro catheters or pipettes so as to be able to accommodate the small vacuum amounts and sizes of such micro tools.

The assembly of this invention may be used for more than one application of several fluids or volumes. The gear assembly may consist of several different diameter gears, attached to, or in contact with each other. This would allow the use of this device to disperse fluids at different ratios and volumes, or to create different or various volumes.

The assembly of this invention may be used to dispense fluids or to create a vacuum to aspirate fluids or tissue. This may be done by assembling the plunger and thumb parts of the assembly and the extensions on these parts on the same or opposite sides of the link assembly. By placing the plunger assemblies in this way, one plunger might withdraw as one plunger advances, therefore allowing the dispersing and intake to occur at the same time.

### BRIEF DESCRIPTION OF THE DRAWINGS

Details of the invention will become more readily apparent from the following detailed description of several embodiments of the invention when taken in conjunction with the accompanying drawings in which:

FIG. 1 is a partially broken away perspective view of one embodiment of an assembly which is formed in accordance with this invention;

FIG. 2 is an exploded perspective view of the assembly of FIG. 1;

FIG. 3 is plan view of the assembly of FIG. 1 which illustrates how the assembly operates;

FIG. 4 is a plan view similar to FIG. 3 showing a multiple bore embodiment of the assembly of this invention;

FIG. 5 is a plan view similar to FIG. 4 which illustrates how the multiple bore embodiment of the assembly operates;

FIG. 6 is a composite view of the assembly of this invention which illustrates how a catheter having a standard leur fitting can be attached to the assembly; and

FIG. 7 is a view similar to FIG. 6 but showing how a micro tool aspiration pipette can be attached to the assembly.

### DETAILED DESCRIPTION OF SPECIFIC EMBODIMENTS OF THE INVENTION

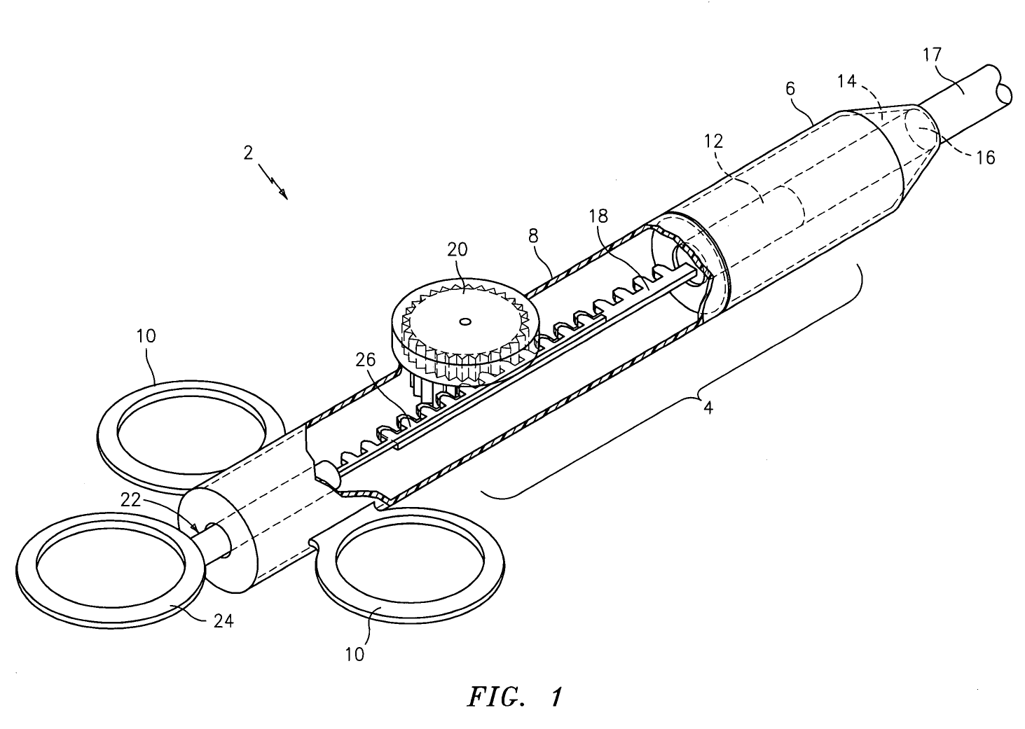

FIG. 1 is a perspective view of one embodiment of a syringe assembly, which is denoted generally by the numeral 2, and which is formed in accordance with this invention. The syringe assembly 2 includes an outer barrel component 4 which may include two interconnected parts 6 and 8, or which may be formed as one unitary part. The barrel 4 may include opposed optional finger rings 10. A plunger 12 is disposed in a bore 14 in the barrel 4. The bore 14 forms an opening 16 at one end of the barrel 4 into which opening 16 an aspirating catheter 17 can be snugly inserted. The plunger 12 is reciprocally movable in the bore 14. A gear toothed rack 18 extends from and is connected to the plunger 12. It will be appreciated that movement of the plunger 12 in the bore 14 is what creates a positive or negative pressure in the bore 14. The assembly 2 also includes a reciprocating thumb piece 22, which may have an optional thumb ring 24, and which has a rack 26 which extends from and is connected to the thumb ring 24. A link 20 in the form of a rotatable gear is mounted in the barrel 4 and engages both of the racks 18 and 26. The assembly 2 operates generally as follows. When the thumb part 22 is pushed or pulled, the gear 20 rotates and causes the plunger 12 to move in the same direction as the thumb part 22, or in the opposite direction from the thumb part 22, as will be explained in greater detail hereinafter.

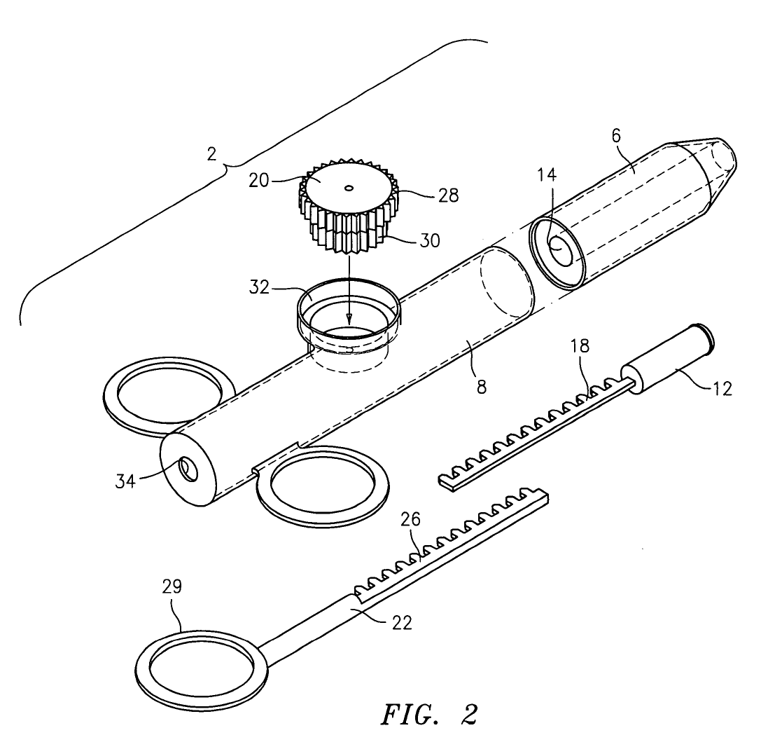

Referring now to FIG. 2, there is shown an exploded view of the syringe assembly 2. The circular gear 20 includes two different diameter parts 28 and 30. The gear 20 fits into a turret 32 in the part 8 of the barrel 4. The larger diameter part 28 of the gear 20 engages the thumb part rack 26 and the smaller diameter part 30 of the gear 20 engages the plunger part rack 18. The thumb part 22 is inserted through an opening 34 in the part 8 of the barrel 4. The plunger part 12 is inserted into the bore 14 in the barrel 4. By providing a multipart gear, such as is shown in FIGS. 1 and 2, the stroke of the thumb part 22 results in a shorter stroke of the plunger 12.

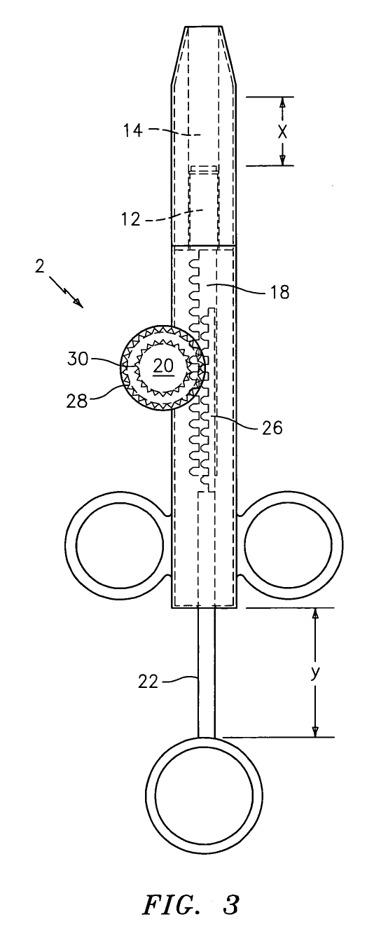

Referring now to FIG. 3, There is shown a typical manner in which the syringe assembly 2 can operate. As shown in FIG. 3, when both of the racks 18 and 26 engage the gear sections 30 and 28 respectively on the same side of the gear 20, retracting movement of the thumb piece 22 will result in retracting movement of the plunger 12. This will cause a negative pressure in the passage 14. The extent of movement of the thumb piece 22 and the plunger 12 will depend on the circumference of the gear section 28 and the gear section 30, respectively. Thus for example, if the circumference of the gear section 28 is twice the circumference of the gear section 30, the distance Y that the thumb part 22 moves will be twice that of the distance X that the plunger 12 moves. It will be appreciated that the degree of negative (or positive) pressure created in the passage 14 will be a function of the distance X that the plunger 12 moves.

As noted above, in the IVF field, when embryos are aspirated into a catheter for replacement into a woman's uterus with a catheter that will be attached to the syringe assembly 2, the negative pressure created in the syringe assembly 2 should be relatively small. Thus, the distance X that the plunger 12 moves should be small. Assume that the distance that the plunger 12 should move in order to create the desired negative pressure is one quarter of an inch. If the ratio of movement of the thumb piece 22 and the plunger 12 were 1:1, then the technician would have to move the thumb piece 22 only one quarter of an inch in order to produce the desire negative pressure in the catheter. In ergonomic reality this small extent of movement of the thumb piece 22 would be very difficult to obtain with any degree of accuracy. Any errors in the extent of movement of the thumb piece 22 would result in the same error in the extent of movement of the plunger 12, and also in an error in the amount of negative pressure created in the passage 14, and in the catheter attached thereto.

Using the syringe assembly of this invention, achievement of the desired extent of movement of the plunger 12 is simplified significantly, and the effect of any errors therein are reduced significantly. If the ratio of the circumference of the gear sections 28 and 30 are, for example, 4:1, then one inch of movement of the thumb piece 22 will result in a one quarter inch movement of the plunger 12, and thus will produce the target negative aspiration pressure in the catheter. Any error in the desired one inch thumb piece movement that may occur will only result in a quarter of that error from the desired one quarter inch plunger movement, thus the pressure in the assembly 2 will deviate minimally from the target desired pressure.

It will be appreciated that movement of the thumb piece 22 in one direction can result in the same direction, or the opposite direction of movement of the plunger 12, depending on whether the respective racks 18 and 26 engage the same side of the gear 20, or opposite sides of the gear 20. If the racks 18 and 26 engage the same side of the gear 20, then retraction of the thumb piece 22 will result in retraction of the plunger 12. If the racks 18 and 26 engage opposite sides of the gear 20, then retraction of the thumb piece 22 will result in forward movement of the plunger 12.

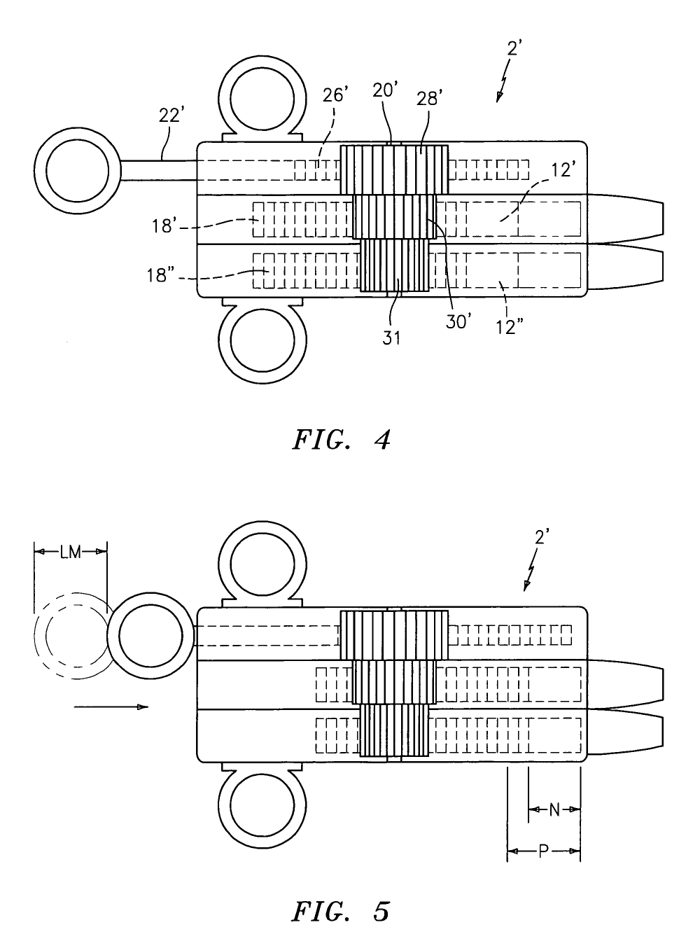

Referring now to FIGS. 4 and 5 there is shown an embodiment of a syringe assembly 2′ which includes a single thumb piece 22′ and a plurality of plungers 12′ and 12″. The gear 20′ has three different diameter toothed portions 28′, 30′ and 31. The thumb piece 22′ includes a toothed rack 26′, and the plungers 12′ and 12″ include respective toothed racks 18′ and 18″. In the embodiment shown in FIGS. 4 and 5, the racks 26′, 18′ and 18″ all engage the same side of the gear 20′, thus forward motion of the thumb piece 22′ will result in forward motion of the plungers 12′ and 12″. Since the racks 18′ and 18″ engage different diameter parts 30′ and 31 of the gear 20′ than the part 28′ that the thumb part rack 26′ engages, the extent of movement of the plungers 12′ and 12″ will be different from each other in that a certain length of movement, or stroke, of the thumb part 22′ will result in a lesser length of movement, or stroke, of the plunger 12′ and a still lesser length of movement, or stroke, of the plunger 12″. Thus, a stroke LM of the thumb part 22′ will result in a shorter stroke P of the plunger 12′ and a still shorter stroke N of the plunger 12″, as shown in FIG. 5. The syringe assembly 2′ can thus be used to expel two different materials in different volumes at the same time with a single stroke of the thumb piece 22′.

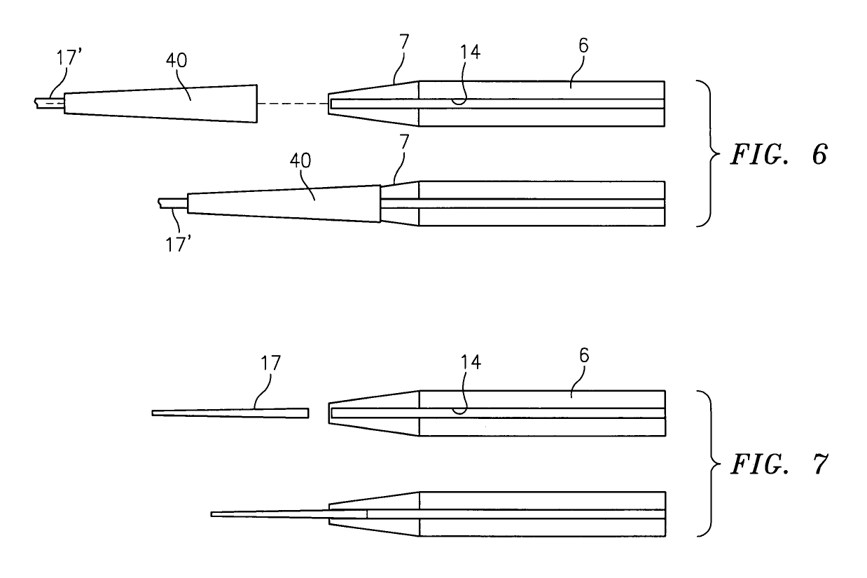

Referring now to FIG. 6, there is shown an embodiment of the frontal portion 6 of the syringe assembly that has a tapered front end 7. The tapered end 7 is configured the same as a luer lock fitting thereby allowing a pipette 40, 17′ to be attached thereto as shown in FIG. 6.

FIG. 7 demonstrates how a micropipette 17 can be connected to the syringe assembly by means of a friction fit between the micropipette 17 and the bore 14 of the assembly.

It will be readily appreciated that the syringe assembly of this invention can be used to aspirate ova into pipettes or catheters a fraction of an inch without requiring that the thumb piece of the syringe be drawn back that same fraction of an inch distance. The assembly minimizes errors in the depth of penetration of the ova in the catheter by enabling an ergonomically large and simple stroke of the thumb piece of the syringe to be translated into a fractionally smaller penetration of the ova into the catheter. The assembly can also be used to expel different materials from the syringe in different volumes, if so desired. For example, in the multibore embodiment described herein, an epoxy and a curing agent for the epoxy could be simultaneously expelled from separate bores in the assembly with a single thumb piece stroke.

Since many changes and variations of the disclosed embodiment of the invention may be made without departing from the inventive concept, it is not intended to limit the invention except as required by the appended claims.

What is claimed is:

1. A syringe assembly comprising:
a) a hollow axially elongated tubular housing;
b) a rotatable cylindrical link mounted in said tubular housing, said rotatable link having at least two different diameter outer portions, one of said portions having a larger diameter than another of said portions whereby the circumference of said one portion is greater than the circumference of said another portion;
c) an axially movable thumb piece mounted on said housing;
d) a first extension connected to said movable thumb piece, said first extension engaging said larger diameter portion of said rotatable link whereby movement of said first extension will cause rotation of said rotatable link;
e) an axially movable plunger mounted in said housing; and
f) a second extension connected to said movable plunger, said second extension engaging said smaller diameter portion of said rotatable link whereby rotation of said rotatable link will cause movement of said second extension and said plunger whereby the extent of movement of said plunger is a predetermined fraction of the extent of concurrent movement of said thumb piece.

2. The syringe assembly of claim 1, wherein said thumb piece and said plunger move in the same direction when said thumb piece is moved.

3. The syringe assembly of claim 1, wherein said thumb piece and said plunger move in opposite directions when said thumb piece is moved.

4. The syringe assembly of claim 1 wherein said different diameter outer portions of said link are provided with gear teeth and said first and second extensions are gear toothed racks which engage said gear teeth.

5. A syringe assembly comprising:
a) a hollow axially elongated tubular housing;
b) a rotatable cylindrical gear mounted in said tubular housing, said rotatable gear having at least two different diameter outer portions which are provided with gear teeth, one of said portions having a larger diameter than another of said portions whereby the circumference of said one portion is greater than the circumference of said another portion;
c) an axially movable thumb piece mounted on said housing;
d) a first gear toothed rack extension connected to said movable thumb piece, said the gear teeth on first extension engaging the gear teeth on said larger diameter portion of said rotatable link whereby movement of said first extension will cause rotation of said rotatable gear;
e) an axially movable plunger mounted in said housing; and
f) a second gear toothed rack extension connected to said movable plunger, the gear teeth on said second extension engaging the gear teeth on said smaller diameter portion of said rotatable gear whereby rotation of said rotatable link will cause movement of said second extension and said plunger whereby movement of said plunger is a predetermined fraction of the extent of concurrent movement of said thumb piece.

6. A dispenser assembly comprising:
a) a hollow axially elongated tubular housing having at least three separate tubular bores;
b) a rotatable gear mounted in said tubular housing, said rotatable gear having at least three outer gear toothed portions, one of said gear toothed portions being disposed in a first one of said tubular bores and having a larger diameter than a others of said gear toothed portions which are disposed in second and third ones of said tubular bores, whereby there are more teeth on said one gear toothed portion than there are on either of said other gear toothed portions;
c) an axially movable thumb piece mounted on said housing;
d) a first gear toothed rack connected to said movable thumb piece and disposed in said first one of said tubular bores, said first toothed rack engaging said larger diameter gear toothed portion of said rotatable gear whereby movement of said first toothed rack will cause rotation of said rotatable gear;
e) axially movable plungers mounted in said second and third bores; and
f) second and third toothed racks connected to said movable plungers, said second and third toothed racks engaging said smaller diameter toothed portions of said rotatable gear whereby rotation of said rotatable gear will cause movement of said second and third toothed racks and said plungers whereby the extent of movement of said plungers is a predetermined fraction of the extent of concurrent movement of said thumb piece.

7. The dispenser assembly of claim 6 wherein the dispenser assembly dispenses an epoxy from one of said tubular bores and concurrently dispenses a curing agent or catalyst material from the other of said tubular bores.

8. A syringe assembly comprising:
a) a hollow axially elongated tubular housing having at least three separate tubular bores;
b) a rotatable gear mounted in said tubular housing, said rotatable gear having at least three outer gear toothed portions, one of said gear toothed portions being disposed in a first one of said tubular bores and having a larger diameter than a others of said gear toothed portions which are disposed in second and third ones of said tubular bores, whereby there are more teeth on said one gear toothed portion than there are on either of said other gear toothed portions;
c) an axially movable thumb piece mounted on said housing;
d) a first gear toothed rack connected to said movable thumb piece and disposed in said first one of said tubular bores, said first toothed rack engaging said larger diameter gear toothed portion of said rotatable gear whereby movement of said first toothed rack will cause rotation of said rotatable gear;
e) axially movable plungers mounted in said second and third bores;
f) second and third toothed racks connected to said movable plungers, said second and third toothed racks engaging said smaller diameter toothed portions of said rotatable gear whereby rotation of said rotatable gear will cause movement of said second and third toothed racks and said plungers whereby the extent of movement of said plungers is a predetermined fraction of the extent of concurrent movement of said thumb piece; and
g) said other gear toothed portions which are disposed in said second and third ones of said tubular bores have different diameters so that one of said other gear toothed portions has a smaller diameter than the other of said gear toothed portions whereby the extent of movement of said plungers are each a different fraction of the extent of movement of said thumb piece.
